(19) United States
(12) Patent Application Publication
(10) Pub. No.: US 2005/0228237 A1
(43) Pub. Date: Oct. 13, 2005

(54) Method for analyzing the biological age of a subject

(76) Inventor: Frank Shallenberger, Carson City, NV (US)

Correspondence Address: KRAMER & AMADO, P.C.;Suite 240, 1725 Duke Street, Alexandria, VA, 22314, US

(21) Appl. No.: 10/822,172

(22) Filed: Apr. 12, 2004

(51) Int. Cl. A61K 31/56; A61B 5/00

(52) U.S. Cl. 600/300; 128/898

(57) ABSTRACT

The present invention is a method for analyzing biological age of a subject. The method analyzes the biological age as it relates to a number of factors indicating levels of health, energy production and metabolism. The method may also be used to calculate a subject's biological age and treat the factors associated with biological age.

### BACKGROUND OF THE INVENTION

1. Field of the Invention

This invention relates generally to a method for analyzing the biological age of a subject and treating the subject as a result of the analysis.

2. Description of Related Art

The concept of a biological age is one that has been discussed in longevity and anti-aging literature for many years. It is a measurement or series of measurements that purport to indicate whether or not an individual is aging rapidly or slowly as compared to their chronological age. An accurate analysis of biological age is needed to allow a medical practitioner to create a program to slow down or decrease the aging of a patient and in turn decrease the patient's biological age.

Numerous attempts have been made to quantify biological age in relation to genetics, ethnicity, lifestyle choices and organ function. However none of the prior methods are a true representation of an individual's biological age. Previous attempts do not incorporate the entire range of factors necessary to analyze biological age. Furthermore, biological age can only be properly understood in the context of human aging generally. Therefore the factors that indicate the status of an individual's biological age must be analyzed with respect to similar situated individuals with similar physical characteristics but different ages to understand the biological age of the individual.

### SUMMARY OF THE INVENTION

In light of the present need for an accurate method of analyzing the biological age of an individual, a brief summary of the present invention is presented. Some simplifications and omission may be made in the following summary, which is intended to highlight and introduce some aspects of the present invention, but not to limit its scope. Detailed descriptions of a preferred exemplary embodiment adequate to allow those of ordinary skill in the art to make and use the invention concepts will follow in later sections.

The present invention includes a method for analyzing the biological age of a subject where the factor of interest is the energy production of a subject. The method comprises: obtaining age, body fat percentage, weight and sex information from a subject; measuring the subject's average oxygen consumption when the subject's respiratory exchange ratio is about 1.0; calculating the subject's predicted maximum oxygen consumption based on the subject's sex, body fat percentage, weight and age in years; wherein the age in years of a subject over a predetermined age is a default age and the age in years of a subject under said predetermined age is the subject's actual age; dividing the subject's average oxygen consumption by the subject's predicted maximum oxygen consumption to obtain an energy production value; and comparing the subject's energy production value to a target energy quotient range for the subject's appropriate age group.

The present invention also includes a method for treating the biological age of a subject comprising: obtaining age, body fat percentage, weight and sex information from a subject; measuring the subject's average oxygen consumption when the subject's respiratory exchange ratio is about 1.0; calculating the subject's predicted maximum oxygen consumption based on the subject's sex, body fat percentage, weight and age in years; wherein the age in years of a subject over a predetermined age is a default age and the age in years of a subject under said predetermined age is the subject's actual age; dividing the subject's average oxygen consumption by the subject's predicted maximum oxygen consumption to obtain an energy production value; comparing the subject's energy production value to a target energy production range for the subject's appropriate age group; and administering a program of nutrition and exercise to said subject to improve said subject's energy production.

The present invention further includes a method for analyzing the biological age of a subject where the factor of interest is the metabolic rate of a subject. The method comprises: obtaining age, height, weight and sex information from a subject; measuring the subject's average resting oxygen consumption; calculating said subject's predicted basal metabolic rate based on the subject's sex, height, weight and age in years; wherein the age in years of a subject over a predetermined age is a default age and the age in years of a subject under said predetermined age is the subject's actual age; dividing said subject's average resting oxygen consumption by said subject's predicted basal metabolic rate to obtain a metabolic rate value for said subject; comparing said metabolic rate value with a target metabolic rate range for said subject's appropriate age group.

The present invention further includes a method for analyzing the biological age of a subject where the factor of interest is the resting fat metabolism of the subject. The method comprises: obtaining age, body fat percentage, weight and sex information from a subject; measuring the subject's resting respiratory exchange ratio; calculating said subject's resting fat metabolism as a function of said subject's resting respiratory exchange ratio; wherein a high resting respiratory exchange ratio indicates excessive dietary carbohydrate ingestion resulting in impaired resting fat metabolism; and a low resting respiratory exchange ratio indicates optimal dietary carbohydrate ingestion resulting in a healthy resting fat metabolism.

The present invention further includes a method for analyzing the biological age of a subject where the factor of interest is the exertional fat metabolism of the subject. The method comprises: obtaining age, body fat percentage, weight and sex information from a subject; measuring said subject's average oxygen consumption when said subject's exertional respiratory exchange ratio is about 0.85; calculating the subject's predicted maximum oxygen consumption based on the subject's sex, body fat percentage, weight and age in years of a subject over a predetermined age is a default age and the age in years of a subject under said predetermined age is the subject's actual age; dividing said subject's average oxygen consumption at exertional respiratory exchange ratio of about 0.85 by said subject's predicted maximum oxygen consumption to obtain an exertional fat metabolism value for said subject; and comparing said subject's exertional fat metabolism value to a target fat metabolism range for the subject's appropriate age group.

The present invention further includes a method for analyzing the biological age of a subject where the factor of interest is the overall fitness of the subject. The method comprises: obtaining age, body fat percentage, weight and sex information from a subject; measuring said subject's average work produced when said subject's exertional respiratory exchange ratio is about 1.00; calculating the subject's predicted maximum oxygen consumption based on the subject's sex, body fat percentage, weight and age in years of a subject over a predetermined age is a default age and the age in years of a subject under said predetermined age is the subject's actual age; calculating said subject's predicted maximum work produced as a function of said subject's predicted maximum oxygen consumption; dividing said subject's average work produced when said subject's exertional respiratory exchange ratio is about 1.00 by said subject's predicted maximum work produced to obtain an overall fitness value for said subject; comparing said subject's overall fitness value to a target overall fitness range for said subject's appropriate age group.

The present invention also includes a method for analyzing the biological age of a subject comprising: obtaining weight, body fat percentage, and sex information from a subject, and measuring said subject's average oxygen consumption when the subject's respiratory exchange rate is about 1.00.

### DETAILED DESCRIPTION OF THE PREFERRED EMBODIMENTS OF THE INVENTION

The present invention describes a method for analyzing the biological age of a subject. This method is required for both analyzing the multiple factors that present an overall view of the biological age of a patient and for treating the various factors to improve the biological age of a patient.

A number of factors make up the total understanding of the biological age of a patient. These factors are determined through a number of measurements taken from the patient by a medical practitioner. The measurements may include both physical characteristics and breath-by-breath measurements. The physical characteristics of the patient may include: the subject's age, height, weight, sex, supine and standing blood pressures, and body fat percentage as determined by a bio-impedance measurement. The breath-by-breath measurements are taken using a specialized device such as a pulmonary gas exchange analyzer. The breath-by-breath measurements may include: oxygen consumption while resting; oxygen consumption during exercise; carbon dioxide production while resting; carbon dioxide production during exercise; work as measured in watts during exercise; heart rate while resting; heart rate during exercise; and respiratory rate while resting.

The breath-by-breath measurements may then be used to prepare a number of data points relating to the factors used for analyzing biological age. These data points may include: average resting oxygen consumption; average resting respiratory exchange ratio; exertional respiratory exchange ratio; average resting heart rate; average resting respiratory rate; average resting end tidal CO2 percentage, average resting carbon dioxide production; anaerobic threshold (the point at which the respiratory exchange ratio is equal to about 1.0 on average); exertional average oxygen consumption at anaerobic threshold; average amount of work in watts at anaerobic threshold, average heart rate at anaerobic threshold; the point at which the patient's respiratory exchange rate is equal to 0.85 on average; average exertional oxygen consumption when the respiratory exchange rate is equal to 0.85; and average amount of work as measured in watts produced when the respiratory exchange rate is equal to 0.85, and average heart rate when the respiratory exchange rate is equal to 0.85.

In order to provide a more exact analysis of the patient's biological age, it is a preferred embodiment of the invention to take breath-by-breath readings according a specific protocol which removes erroneous information. The preferred embodiment includes recording all breath samplings, respiratory rate and heart rate measurements for a continuous interval of seven minutes while the subject is at rest. The best five of seven averages are computed and recorded every 15 second interval for heart rate, respiratory rate, respiratory exchange ratio, and average oxygen consumption. The readings taken during the first minute are rejected. The remaining readings are designated as resting readings. All resting average oxygen consumption readings greater than a predicted BMR divided by 5.5 are removed, and all values less than predicted BMR divided by 11 are removed. The predicted BMR is calculated depending on sex. For men the predicted BMR=66.4730+(13.7516×weight)+(5.0033×Height)−(6.7550×Age). For women the predicted BMR=655.0950+(9.536×weight)+(1.8496×height)−(4.6756×Age). After resting average oxygen consumption readings have been removed based on the subject's corresponding BMR, the remaining two highest and lowest values are removed. The remaining values are designated as “remaining resting oxygen consumption readings.” The remaining resting oxygen consumption readings are used to determine the following measuring points:

Resting Average Oxygen Consumption (RVO2): The resting average oxygen consumption is determined by the equation: RVO2=((2×lowest remaining resting oxygen consumption reading)+highest remaining resting oxygen consumption reading)/3

Resting Respiratory Exchange Ratio (RRER): The resting respiratory exchange ratio is the average ratio of carbon dioxide produced to oxygen consumed while the subject is at rest. In a preferred embodiment of the invention any remaining resting oxygen consumption readings that correspond to respiratory exchange readings greater than 0.95 and less than 0.72 are removed in addition to those readings removed as shown above. The average of the remaining respiratory exchange readings corresponding to the now remaining resting oxygen consumption readings gives the average resting respiratory exchange ratio.

In another preferred embodiment of the invention, a protocol for removing erroneous data is also applied to the breath-by-breath readings taken while the subject is exercising. In the preferred embodiment, the best five of seven averages are computed and recorded every 15 second interval for heart rate, respiratory exchange ratio and average oxygen consumption. After ignoring the first minute of data points, a fat burning heart rate sample range is determined between a minimum respiratory exchange rate greater than or equal to 0.82 and a maximum respiratory exchange rate less than or equal to 0.88. When there is no respiratory exchange ratio less than or equal to 0.88, the maximum respiratory exchange ratio is equal to the lowest respiratory exchange ratio reading. The fat burning heart rate is one half of the sum of the highest heart rate and the lowest heart rate in the fat burning heart rate sample range. The fat burning work range is determined between a first fat burning heart rate when approaching from the lower numbers greater than or equal to the fat burning heart rate −2 and a first fat burning heart rate when approaching from the higher numbers less than or equal to the fat burning heart rate +2. The anaerobic threshold heart rate sample range is determined between a minimum second respiratory exchange rate greater than or equal to 0.98 and a maximum second respiratory exchange rate less than or equal to 1.02. The anaerobic threshold heart rate is one half of the sum of the highest heart rate and the lowest hear rate in the anaerobic threshold heart rate sample range. The anaerobic threshold heart rate work range is determined between a minimum first heart rate of greater than or equal to anaerobic threshold heart rate −2 and a maximum first heart rate less than or equal to anaerobic threshold heart rate +2.

In a preferred embodiment of the invention, the data points determined above are further used to calculate the following measurements:

The subject's average oxygen consumption when the when the subject is exerting at a respiratory exchange rate of about 0.85 (RER.85VO2) is equal to one half of the sum of the highest and lowest oxygen consumption values found in the fat burning heart rate work range.

The subject's average work produced (as measure in watts) when the subject is exerting at a respiratory exchange rate of about 0.85 (RER.85WORK) is equal to one half of the sum of the highest and lowest work values found in the fat burning heart rate work range.

The subject's average oxygen consumption when the subject is exerting at a respiratory exchange ratio of about 1.00 or anaerobic threshold (ATVO2) is equal to one half of the sum of the highest and lowest average oxygen consumptions in the anaerobic threshold heart rate range.

The subject's average amount of work produced (as measure in watts) when the subject is exerting at a respiratory exchange ratio of about 1.00 or anaerobic threshold (ATWORK) is equal to the one half of the sum of the highest and lowest work values found in the anaerobic threshold heart rate work range.

These data points are then used to assess the factors relating to the biological age of the subject. These factors include, but are not limited to the energy production of the subject, the basal metabolic rate of the subject, the resting fat metabolism of the subject, the exertional fat metabolism of the subject, the work fat metabolism of the subject, the overall fitness of the subject and the biological age of the subject.

In one embodiment of the invention, the method includes analyzing the biological age of a subject as it relates to the energy production of the subject. In a preferred embodiment, the energy production of the subject is equal to the average oxygen consumption of the subject at a respiratory exchange ratio of about 1.00 divided by the predicted maximum oxygen consumption for the subject.

The predicted maximum oxygen consumption for a person is determined based on the age, sex, weight, and body fat percentage of the patient as follows: predicted maximum average oxygen consumption for a male=[weight (in kilograms)×(1−(body fat percentage/100))/0.82]×(50.72−(0.372×age)). Predicted maximum average oxygen consumption for a female=([weight (in kilograms)×(1−(body fat percentage/100))/0.78]+43)×(22.78−(0.17×age)).

Accurately analyzing the biological age of a subject requires taking the equations for calculating predicted maximum oxygen consumption and adjusting them for age. In a preferred embodiment of the invention, when calculating predicted maximum oxygen consumption for a subject whose actual age in years is over a predetermined age, a default age is used instead. In a more preferred embodiment of the invention, the predetermined age is 35-60. In the most preferred embodiment, the predetermined age is forty (40). The predetermined age is chosen as the age at which individuals generally first begin to show signs of biological aging. Therefore if the general population begins to age either more quickly or more slowly, the predetermined age may also change accordingly. The predetermined age and default age may be different. In the most preferred embodiment of the invention, the predetermined age is equal to the default age. In another preferred embodiment, when calculating predicted maximum oxygen consumption for a subject whose actual age is under forty (40), the actual age of the subject is used. The most preferred embodiment may be illustrated by the following examples: For a subject with actual age greater than the predetermined age: Subject's actual age is 50, greater than the predetermined age of 40, a default age of 40 is used to calculate the subject's predicted maximum oxygen consumption. For a subjection with actual age less than the predetermined age: Subject's actual age is 35, lower than the predetermined age of 40, the subject's actual age of 35 is used to calculate the subject's predicted maximum oxygen consumption.

Once the average oxygen consumption at anaerobic threshold of an individual is divided by the individual's predicted maximum oxygen consumption, the number is multiplied by 166.66 to assess the individual's energy production value. An energy production value greater than 100 is the goal of every patient. The goal of patients under fifty years old is to achieve and energy production value greater than 120. Persons older than 60 to 70 years of age should target an energy production value greater than 100.

Accurately assessing the energy production value of a subject allows a practitioner to begin to treat and reduce the biological age of the subject. Therefore, a further embodiment of the invention includes treating the biological age of a subject. Low energy production can result from a number of factors related to the biological aging of the subject. These factors may include disease, nutrition and exercise. In particular, a physician may treat heart, lung and breathing related disease using conventional means to improve the patient's energy production. In a preferred embodiment of the invention, a physician may also prepare and administer a nutrition and exercise program to a patient in order to improve energy production. In a more preferred embodiment, a nutrition program may include decreased caloric intake, decrease dietary carbohydrate intake, nutritional supplementation, hormonal replacement, therapeutic detoxification, and medication. In another more preferred embodiment, an exercise program may include zone interval training and or zone circuit training, wherein the levels described as “regular intensity” and “high intensity” are determined by the subject's energy production measurements. Zone interval training is defined as an exercise regimen where a subject exercises at regular intensity then intersperses intervals of high intensity exercise for a predetermined period of time. Zone circuit training combines interval training with multiple repetitions of resistance training exercise for a predetermined period of time. In a preferred embodiment of the invention, periods for zone interval training and zone circuit training are used to improve a subject's energy production. Furthermore, both the nutritional program and exercise program may be combined to maximize treatment of a patient's energy production.

In another preferred embodiment of the invention, the method is used to analyze the biological age of a subject realizing that the biological age of a subject is influenced by a multiplicity of factors. In order to understand why the biological age is what it is, and what treatment measures must be initiated in order to improve the biological age, these other factors must be further determined as follows.

In another preferred embodiment of the invention, the method is used to analyze the biological age of a subject as it relates to the basal metabolic rate of the subject. The basal metabolic rate value is determined by dividing the subject's average resting oxygen consumption by the predicted basal metabolic rate of the subject.

In a preferred embodiment of the invention, the predicted basal metabolic rate is calculated based on the sex of the subject. In a further preferred embodiment the predicted metabolic rate for males is equal to 66.4730+(13.7516×weight)+(5.0033×height)−(6.7550×age). In another further preferred embodiment the predicted metabolic rate for females is equal to 655.0950+(9.536×weight)+(1.8496×height)−(4.6756×age). In a preferred embodiment of the invention, when calculating predicted basal metabolic rate for a subject whose actual age in years is over a predetermined age, a default age is used instead. In a more preferred embodiment of the invention, the predetermined age is 35-60. In the most preferred embodiment, the predetermined age is 40. The predetermined age is chosen as the age at which individuals generally first begin to show signs of biological aging. Therefore if the general population begins to age either more quickly or more slowly, the predetermined age may also change accordingly. The predetermined age and default age may be different. In the preferred embodiment of the invention, the predetermined age is equal to the default age. In another preferred embodiment, when calculating predicted basal metabolic for a subject whose actual age is under forty, the actual age of the subject is used.

In the preferred embodiment, the equation for calculating metabolic rate value is equal to 6.95×average resting oxygen consumption (RVO2) multiplied by 100 and divided by the predicted metabolic rate. In a further preferred embodiment, the target range for a subject's metabolic rate value is between 90 and 110. A metabolic rate value in this range shows optimal metabolic rate and results in a lower biological age. A metabolic rate value below this range shows a low metabolic rate and results in an increased biological age. A metabolic rate value below the target range may also be indicative a number of other health deficiencies or other issues including but not limited to: adrenal insufficiency, thyroid deficiency, insufficient sleep, deficient muscle mass, testosterone deficiency, growth hormone deficiency, nutritional deficiency, excessive estrogen, progesterone deficiency, and dehydration. A metabolic rate >110 may indicate a hyper-metabolic condition including but not limited to hyperthyroidism, pain, fever, and certain disease states.

Another embodiment of the invention includes analyzing biological age as it relates to the resting fat metabolism of a subject. In a preferred embodiment of the invention, the resting fat metabolism of a subject is analyzed as a function of the subject's resting respiratory exchange ratio (RRER). In the most preferred embodiment, the resting fat metabolism is equal to 220.44−(resting respiratory exchange ratio×167).

In a preferred embodiment of the invention a calculated resting fat metabolism of greater than 90 indicates optimal fat metabolism which results in decreased biological age. Alternatively, a resting fat metabolism of less than 90 is indicative of sub-optimal fat metabolism which results in increased biological age. In further preferred embodiment of the invention, sub-optimal resting fat metabolism and increased biological age are treating by prescribing a diet restricting carbohydrates and increasing nutritional supplementation. Extreme cases of low resting fat metabolism may require therapeutic detoxification, hormonal replacement and/or other medical intervention.

In another preferred embodiment of the invention, the method is used to analyze biological age as it relates to the exertional fat metabolism of a subject. In a further preferred embodiment, exertional fat metabolism of a subject is calculated by dividing the subject's average oxygen consumption when the subject's exertional respiratory rate is about 0.85 by the subject's predicted maximum oxygen consumption based on the subject's sex, height, weight and age in years. The subject's predicted maximum oxygen consumption is calculated as shown above in the section for calculating the subject's energy production.

In a further preferred embodiment of the invention, the subject's exertional fat metabolism value is calculated as (333.33×average oxygen consumption when the subject's exertional respiratory rate is about 0.85)/(predicted maximum oxygen consumption). In a preferred embodiment of the invention a target fat metabolism range is 90 to 100. In a further preferred embodiment of the invention, an exertional fat metabolism value of greater than or equal to 100 indicates optimal exertional fat metabolism which results in decreased biological age. In another further preferred embodiment of the invention, an exertional fat metabolism value of less than 100 indicates decreasing exertional fat metabolism which results in increased biological age. An exertional fat metabolism value significantly less than 90 may indicate diabetes, insulin resistance, excessive carbohydrate intake, hormonal deficiencies, excessive trans fatty acids, nutritional deficiencies and/or additional health deficiencies. The further preferred embodiment of the invention may include treating decreased exertional fat metabolism by prescribing nutritional supplementation, dietary restrictions, and/or other medical treatments.

Another embodiment of the invention includes a method analyzing the biological age of a subject as it relates to the work fat metabolism of a subject. In a preferred embodiment, the work fat metabolism of a subject is equal to the subject's average work produced divided by the subject's predicted maximum average work produced.

In a preferred embodiment of the invention, the subject's predicted maximum work produced for a male is equal to (predicted maximum oxygen consumption −(5.8×[weight (in kilograms)×(1−(body fat percentage/100))/0.82])−151)/10.1 and for a female is equal to (predicted maximum oxygen consumption−(5.8×[weight (in kilograms)×(1−(body fat percentage/100))/0.78)])−151)/10.1. The predicted maximum oxygen consumption is calculated as shown above.

In a preferred embodiment of the invention, the work fat metabolism value is equal to (the average work produced when said subject's exertional respiratory exchange ratio is about 0.85×200) divided by predicted maximum work produced. In a preferred embodiment of the invention, the target range for a subject's work fat metabolism is 90 to 100. In a more preferred embodiment of the invention, a work fat metabolism value greater than or equal to 100 indicates optimal work fat metabolism which results in decreased biological age. In another more preferred embodiment, a work fat metabolism value less than 100 indicates decreasing work fat metabolism which results in increasing biological age. A work fat metabolism value significantly lower than the target range and 10 to 20 points lower than the exertional fat metabolism target may indicate decreased muscle mass or sarcopenia. The further preferred embodiment of the invention may include a method for treating decreased work fat metabolism by prescribing nutritional supplementation, hormonal replacement, dietary restriction, and an exercise regimen, where the exercise regimen may include weight resistance training and/or aerobic training.

Another embodiment of the invention includes a method for analyzing biological age as a factor of a subject's overall fitness. In a preferred embodiment of the invention, the subject's overall fitness is calculated as the subject's average work produced when the subject's exertional respiratory rate exchange ratio is about 1.00 divided by the subject's predicted maximum work produced as a function of the subject's predicted maximum oxygen consumption.

In a further preferred embodiment of the invention, the subject's overall fitness value is equal to (the subject's average work produced when the subject's exertional respiratory rate exchange ratio is about 1.00×125) divided by the subject's predicted maximum work produced. The subject's predicted maximum work produced is described in detail above.

In a preferred embodiment of the invention, a target overall fitness range is 90 to 100. In a further preferred embodiment, an overall fitness value of greater than or equal to 100 indicates optimal strength and fitness which results in decreased biological age. In another preferred embodiment of the invention, an overall fitness value of less than 100 indicates decreased strength and fitness which results in increased biological age. An overall fitness value significantly lower than 100 and 20 points lower than the subject's energy production value may indicate decreased muscle mass or sarcopenia. The preferred embodiment of the invention may include a method for treating decreased strength and fitness by prescribing nutritional supplementation, hormonal replacement, and an exercise regimen. The exercise regimen may include resistance training and/or aerobic exercise.

In another embodiment of the invention, the medical practitioner analyzes the subject's biological age by directly calculating the biological age as it relates to the biological aging process of the individual. In a preferred embodiment of the invention, a male subject's biological age is calculated as follows: biological age=136−(1.66×the subject's average oxygen consumption at anaerobic threshold average)/(0.372×[weight (in kilograms)×(1−(body fat percentage/100))/0.82]). A female subject's biological age is calculated as follows: biological age=134−(1.66× the subject's average oxygen consumption at anaerobic threshold average)/(0.17×([weight (in kilograms)×(1−(body fat percentage/100))/0.78]+43). By applying this equation, the practitioner can see that an individual with a predicted maximum oxygen consumption at anaerobic threshold greater than their actual average oxygen consumption at anaerobic threshold will have a biological age greater than their chronological age, while an individual with a predicted maximum oxygen consumption at anaerobic threshold less than their actual average oxygen consumption at anaerobic threshold will have a biological age less than their chronological age.

In a further preferred embodiment, a medical practitioner may prescribe a program of nutrition and exercise to decrease the biological age of the subject, where the subject's biological age is greater than their chronological age. The program is determined by the other metabolic factors as described and calculated above and may include nutritional supplementation, dietary restrictions, hormonal replacement, medication, and a specified exercise regimen targeting the factors calculated above. The exercise regimen may include resistance training and/or aerobic training.

Although the present invention has been described in detail with particular reference to preferred embodiments thereof, it should be understood that the invention is capable of other different embodiments, and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only, and do not in any way limit the invention, which is defined only by the claims.

1. A method for analyzing the biological age of a subject comprising:
obtaining age, body fat percentage, weight and sex information from a subject;
measuring said subject's average oxygen consumption when the subject's respiratory exchange rate is about 1.0;
calculating the subject's predicted maximum oxygen consumption based on the subject's sex, body fat percentage, weight and age in years;
wherein the age in years of a subject over a predetermined age is a default age and the age in years of a subject under said predetermined age is the subject's actual age;
dividing said subject's average oxygen consumption by said subject's predicted maximum oxygen consumption to obtain an energy production value for said subject; and
comparing said subject's energy production to a target energy production range for said subject's appropriate age group.

2. The method of claim 1, wherein the predetermined age is equal to the default age.

3. The method of claim 1 further comprising, treating said subject to improve said subject's energy production in relation to the subject's appropriate age range.

4. The method of claim 3, further comprising administering a program of nutrition and exercise to the subject to improve said subject's energy production.

5. The method of claim 1 further comprising, applying said subject's energy production to assess the subject's biological age.

6. The method of claim 5 further comprising, calculating an average predicted energy production using data from individuals of different ages having the same sex, height and weight as the subject.

7. The method of claim 6, wherein a subject with an energy quotient higher than the average predicted energy production for said subject has a biological age lower than said subject's actual age.

8. The method of claim 6, wherein a subject with an energy production lower than the average predicted energy production for said subject has a biological age higher than said subject's actual age.

9. A method for treating the biological age of a subject comprising:
obtaining age, body fat percentage, weight and sex information from a subject;
measuring said subject's average oxygen consumption when the subject's respiratory exchange rate is about 1.0;
calculating the subject's predicted maximum oxygen consumption based on the subject's sex, body fat percentage, weight and age in years;
wherein the age in years of a subject over a predetermined age is a default age and the age in years of a subject under said predetermined age is the subject's actual age;
dividing said subject's average oxygen consumption by said subject's predicted maximum oxygen consumption to obtain an energy production value for said subject;
comparing said subject's energy production to a target energy production range for said subject's appropriate age group; and
administering a program of nutrition and exercise to said subject to improve said subject's energy production.

10. The method of claim 9, wherein the predetermined age is equal to the default age.

11. The method of claim 9 further comprising, applying said subject's energy production to assess the subject's biological age.

12. The method of claim 11 further comprising, calculating a predicted energy production using data from individuals of different ages having the same sex, height and weight as the subject.

13. The method of claim 12, wherein a subject with an energy production value higher than the predicted energy production for said subject has a biological age lower than said subject's actual age.

14. The method of claim 12, wherein a subject with an energy production value lower than the predicted energy production for said subject has a biological age higher than said subject's actual age.

15. A method for analyzing the biological age of a subject comprising:
obtaining age, body fat percentage, weight and sex information from a subject;
measuring the subject's average resting oxygen consumption;
calculating said subject's predicted basal metabolic rate based on the subject's sex, body fat percentage, weight and age in years;
wherein the age in years of a subject over a predetermined age is a default age and the age in years of a subject under said predetermined age is the subject's actual age;
dividing said subject's average resting oxygen consumption by said subject's predicted basal metabolic rate to obtain a metabolic rate value for said subject;
comparing said metabolic rate value with a target metabolic rate range for said subject's appropriate age group.

16. The method of claim 15, wherein the predetermined age is equal to the default age.

17. The method of claim 15 further comprising, treating said subject to improve said subject's metabolic rate in relation to the subject's appropriate age range.

18. The method of claim 17 further comprising, administering a program of nutrition and exercise to the subject to improve said subject's metabolic rate.

19. The method of claim 15 further comprising, applying said subject's metabolic rate to assess the subject's biological age.

20. The method of claim 19, wherein a metabolic rate value lower than the target metabolic rate range for said subject's appropriate age group indicates increased biological age.

21. The method of claim 19, wherein a metabolic rate value higher than the target metabolic rate range for said subject's appropriate age group indicates decreased biological age.

22. The method of claim 19, wherein a metabolic rate value lower than the target metabolic rate range indicates adrenal insufficiency, thyroid deficiency, insufficient sleep, deficient muscle mass, testosterone deficiency, growth hormone deficiency, nutritional deficiency, excessive estrogen, progesterone deficiency, dehydration, inflammatory illness, anxiety, invalid test results or a combination thereof.

23. A method for analyzing the biological age of a subject comprising;
obtaining age, body fat percentage, weight and sex information from a subject;
measuring the subject's resting respiratory exchange ratio;
calculating said subject's fat metabolism as a function of said subject's resting respiratory exchange ratio;
wherein a low fat metabolism as a function of said subject's respiratory resting exchange ratio indicates increased carbohydrate metabolism and impaired fat metabolism;
and a high fat metabolism as a function of said subject's resting respiratory exchange ratio indicates decreased carbohydrate metabolism and healthy fat metabolism.

24. The method of claim 23, wherein low fat metabolism as a function of said subject's resting respiratory exchange ratio indicates increased carbohydrate intake.

25. The method of claim 23, wherein low fat metabolism as a function of said subject's resting respiratory exchange ratio indicates increased biological age.

26. The method of claim 25 further comprising, decreasing biological age by prescribing a diet of reduced carbohydrate consumption and nutritional supplementation.

27. The method of claim 24 further comprising, prescribing a diet of reduced carbohydrate consumption and nutritional supplementation.

28. The method of claim 23, wherein high fat metabolism as a function of said subject's resting respiratory exchange ratio indicates decreased biological age.

29. A method for analyzing the biological age of a subject comprising;
obtaining age, body fat percentage, weight and sex information from a subject;
measuring said subject's average oxygen consumption when said subject's exertional respiratory exchange ratio is about 0.85;
calculating the subject's predicted maximum oxygen consumption based on the subject's sex, body fat percentage, weight and age in years;
wherein the age in years of a subject over a predetermined age is a default age and the age in years of a subject under said predetermined age is the subject's actual age;
dividing said subject's average oxygen consumption at exertional respiratory exchange ratio of about 0.85 by said subject's predicted maximum oxygen consumption to obtain an exertional fat metabolism value for said subject; and
comparing said subject's exertional fat metabolism value to a target fat metabolism range for the subject's appropriate age group.

30. The method of claim 29, wherein the predetermined age is equal to the default age.

31. The method of claim 29, wherein a fat metabolism value equal to or higher than the target fat metabolism range indicates optimal fat metabolism.

32. The method of claim 29, wherein a fat metabolism value lower than the target fat metabolism range indicates decreased fat metabolism.

33. The method of claim 32, wherein a fat metabolism value significantly lower than the target fat metabolism range indicates diabetes, insulin resistance, excessive carbohydrate intake, hormonal deficiencies, sleep deficiency, carnitine deficiency, Coenzyme Q10 deficiencies, dietary fat deficiencies, excessive trans fatty acids, nutritional deficiencies or a combination thereof.

34. The method of claim 32 further comprising, treating the subject's fat metabolism by prescribing nutritional supplementation and dietary restrictions.

35. The method of claim 29, wherein a fat metabolism value greater than the target fat metabolism range indicates decreased biological age.

36. The method of claim 29, wherein a fat metabolism value lower than the fat metabolism range indicates increased biological age.

37. A method for analyzing the biological age of a subject comprising;
obtaining age, body fat percentage, weight and sex information from a subject;
measuring said subject's average work produced when said subject's exertional respiratory exchange ratio is about 0.85;
calculating the subject's predicted maximum oxygen consumption based on the subject's sex, body fat percentage, weight and age in years;
wherein the age in years of a subject over a predetermined age is a default age and the age in years of a subject under said predetermined age is the subject's actual age;
calculating said subject's predicted maximum work produced as a function of said subject's predicted maximum oxygen consumption;
dividing said subject's average work produced when said subject's exertional respiratory exchange ratio is about 0.85 by said subject's predicted maximum work produced to obtain a work fat metabolism value for said subject;
comparing said subject's work fat metabolism to a target work fat metabolism range for said subject's appropriate age group.

38. The method of claim 37, wherein the predetermined age is equal to the default age.

39. The method of claim 37, wherein a work fat metabolism equal to or higher than the target work fat metabolism range indicates optimal fat metabolism.

40. The method of claim 37, wherein a work fat metabolism lower than the target work fat metabolism range indicates decreased fat metabolism.

41. The method of claim 40, wherein a work fat metabolism significantly lower than the target work fat metabolism range indicates decreased muscle mass.

42. The method of claim 41 further comprising, treating decreased fat metabolism capability by prescribing nutritional supplementation and an exercise regimen.

43. The method of claim 42, wherein the exercise regimen further comprises weight resistance training.

44. The method of claim 36, wherein a work fat metabolism higher than the target work fat metabolism range indicates decreased biological age.

45. The method of claim 36, wherein a work fat metabolism lower than the target work fat metabolism range indicates increased biological age.

46. A method for analyzing the biological age of a subject comprising:
obtaining age, body fat percentage, weight and sex information from a subject;
measuring said subject's average work produced when said subject's exertional respiratory exchange ratio is about 1.00;
calculating the subject's predicted maximum oxygen consumption based on the subject's sex, body fat percentage, weight and age in years;
wherein the age in years of a subject over a predetermined age is a default age and the age in years of a subject under said predetermined age is the subject's actual age;
calculating said subject's predicted maximum average work produced as a function of said subject's predicted maximum average oxygen consumption;
dividing said subject's average work produced when said subject's exertional respiratory exchange ratio is about 1.00 by said subject's predicted maximum work produced to obtain an overall fitness value for said subject;
comparing said subject's overall fitness value to a target overall fitness range for said subject's appropriate age group.

47. The method of claim 46, wherein the predetermined age is equal to the default age.

48. The method of claim 46, wherein an overall fitness value equal to or higher than the target overall fitness level indicates optimal strength and fitness.

49. The method of claim 46, wherein an overall fitness lower than the target overall fitness range indicates decreased strength and fitness.

50. The method of claim 49, wherein an overall fitness value significantly lower than the target overall fitness range indicates decreased muscle mass.

51. The method of claim 50 further comprising, treating decreased strength and fitness by prescribing nutritional supplementation and an exercise regimen.

52. The method of claim 51, wherein the exercise regimen further comprises weight resistance training.

53. The method of claim 46, wherein an overall fitness value higher than the target work fat metabolism range indicates decreased biological age.

54. The method of claim 46, wherein an overall fitness value lower than the target work fat metabolism range indicates increased biological age.

55. A method for analyzing the biological age of a subject comprising:
obtaining age, body fat percentage, weight and sex information from a subject;
measuring said subject's average oxygen consumption when the subject's respiratory exchange rate is about 1.0;
calculating the subject's biological age as a function of said subject's average oxygen consumption when the subject's respiratory exchange rate is about 1.0;
wherein said calculation is dependent on the sex of the subject;
wherein said calculation quantifies the biological age of a person as a number.

56. The method of claim 55 further comprising, prescribing a program of nutrition and exercise to decrease the biological age of the subject.
